(19) United States
(12) Patent Application Publication
(10) Pub. No.: US 2012/0330212 A1
(43) Pub. Date: Dec. 27, 2012

(54) Magnetically-Triggered Bandage Release Mechanism

(75) Inventors: Joseph T. Persyn, Lakehills, TX (US); Michael J. Rubal, Lytle, TX (US); Robert Windschauer, Tampa, FL (US); James S. Weston, Radford, VA (US)

(73) Assignee: WESTPATCH, LLC, Tampa, FL (US)

(21) Appl. No.: 13/530,885

(22) Filed: Jun. 22, 2012

Related U.S. Application Data
(60) Provisional application No. 61/500,044, filed on Jun. 22, 2011.

(51) Int. Cl. A61F 13/02; B82Y 5/00

(52) U.S. Cl. 602/54; 977/773; 977/931

(57) ABSTRACT

A pressure-sensitive adhesive bandage with a magnetically-released triggering mechanism is provided. The pressure-sensitive bandage may include a backing for providing support for the pressure-sensitive adhesive bandage. Additionally, the pressure-sensitive bandage may include a pressure-sensitive adhesive applied to the backing, which may include microcapsules. The microcapsules may include a silica-based shell, a core confined within the silica-based shell that includes oil, and a plurality of magnetic particles that may be positioned in at least one of the silica-based shell and the core. The plurality of magnetic particles may be configured to cause the silica-based shell to release the oil from the core when a magnetic force is exerted on the plurality of magnetic particles. When the oil is released from the core and contacts the pressure-sensitive adhesive, it causes a decrease in peel strength of the pressure-sensitive adhesive and enables removal of the pressure-sensitive adhesive bandage from a surface.

### CROSS REFERENCE TO RELATED APPLICATIONS

This patent application claims the benefit of U.S. Provisional Patent Application No. 61/500,044, filed Jun. 22, 2011, which is incorporated by reference in its entirety.

### FIELD OF THE INVENTION

The present application relates to bandages, tapes, and bandage and tape removal mechanisms, and, more particularly, to a magnetically-triggered bandage release mechanism.

### BACKGROUND

Bandages, tapes, and other similar types of products are utilized to provide support, prevent bacterial infections, serve as dressings, attach various objects together, and perform a variety of other functions. Most bandages, including pressure-sensitive adhesive bandages, are designed for either permanent or removable applications. Permanent bandages and adhesives may be designed to be initially removable, however, they may be configured to build adhesion forming a permanent bond after several hours or days. On the other hand, removable adhesives are typically designed to form a temporary bond, and may be removed after months or years without leaving residues. Currently, various different types of bandage technologies exist. For example, in addition to traditional bandages other types of bandages include bandages featuring side chain crystalline polymers (SCCPs) used with pressure-sensitive adhesives, bandages that use adhesives that incorporate photo-curing with ultraviolet light and bandages that use solvent additives.

### SUMMARY

A pressure-sensitive adhesive bandage including a magnetically-triggered release mechanism and methods for using the bandage are disclosed. The bandage may be utilized in a variety of situations. For example, the bandage may be utilized as a medical bandage for covering up a wound on the skin of a patient. However, removing traditional bandages from a patient's skin often causes significant pain, irritation, or other unwanted problems. The bandage and methods disclosed herein provide for a magnetically-triggered release mechanism for reducing pain, irritation, or other problems associated with removing a bandage. Specifically, the bandage may include a backing and a pressure-sensitive adhesive applied to the backing. The pressure-sensitive adhesive may include microcapsules that include silica-based shells, cores confined by the silica-based shells that contain oil, and magnetic particles that reside in the shells and/or the cores. When a magnetic force is applied to the magnetic particles, the silica-based shells can release the oil from the core, thereby allowing the oil to contact the pressure-sensitive adhesive. Once the oil contacts the pressure-sensitive adhesive, the peel strength of the pressure-sensitive adhesive may be reduced, which can allow a user to readily remove the bandage from the user's skin.

In one embodiment, a pressure-sensitive adhesive bandage may be provided. The pressure-sensitive bandage may include a backing for providing support for the bandage. Additionally, the bandage may include a pressure-sensitive adhesive that may be applied to the backing. The pressure-sensitive adhesive may include a plurality of microcapsules that may include a silica-based shell, a core confined within the silica-based shell that includes an amount of oil, and a plurality of magnetic particles. The plurality of magnetic particles may be positioned in at least one of the silica-based shell and the core. Also, the plurality of magnetic particles may be configured to cause the silica-based shell to release the oil from the core when a magnetic force is exerted on the plurality of magnetic particles. The oil released from the core may enable removal of the bandage from a surface by causing a decrease in peel strength of the pressure-sensitive adhesive.

In another embodiment, a method for utilizing a pressure-sensitive adhesive bandage may be provided. The method may include positioning applying the bandage to a surface. The pressure-sensitive bandage may include a pressure-sensitive adhesive that may include a plurality of microcapsules. The plurality of microcapsules may include a silica-based shell, a core confined within the silica-based shell that includes an amount of oil, and a plurality of magnetic particles that may be positioned in at least one of the silica-based shell and the core. Additionally, the method may include applying a magnetic force to the plurality of magnetic particles in order to cause the silica-based shell to release the oil from the core. The oil released from the core may cause a decrease in peel strength of the pressure-sensitive adhesive when the oil contacts the pressure-sensitive adhesive. Furthermore, the method may include removing the bandage from the surface after the oil released from the core causes the decrease in peel strength of the pressure-sensitive adhesive.

According to another exemplary embodiment, pressure-sensitive adhesive bandage kit may be provided. The bandage kit may include a backing and a pressure-sensitive adhesive that may be applied to the backing. The pressure-sensitive adhesive may include a plurality of microcapsules that may include a silica-based shell, a core confined within the silica-based shell that includes an amount of oil and a plurality of magnetic particles. The plurality of magnetic particles may be positioned in at least one of the silica-based shell and the oil core. Additionally, the plurality of magnetic particles may be configured to cause the silica-based shell to release the oil from the core when a magnetic force is exerted on the plurality of magnetic particles. The oil released from the core may cause a decrease in peel strength of the pressure-sensitive adhesive when the oil contacts the pressure-sensitive adhesive, thereby allowing the bandage to be removed from a surface.

These and other features are described in the following detailed description, drawings, and appended claims.

### BRIEF DESCRIPTION OF THE DRAWINGS

FIG. 1 is a back view of a bandage featuring a pressure-sensitive adhesive including a magnetically-triggered release mechanism according to an embodiment of the application.

FIG. 2 is a front view of the bandage of FIG. 1.

FIG. 3 is a back view of a bandage including a release liner for protecting the pressure sensitive adhesive of the bandage before use.

FIG. 4 is a view of the silica-based shells and oil-filled cores that include magnetic particles that are utilized with the pressure-sensitive adhesive of a bandage.

FIG. 5 is a view of a silica-based shell that includes magnetic particles and an oil-filled core that is utilized with the pressure-sensitive adhesive of a bandage.

FIG. 6 illustrates the effects on the silica-based shell from the application of a magnetic force applied to the magnetic particles of the pressure-sensitive adhesive of a bandage.

FIG. 7 is a graph of average peel test results for bandages having microparticles with and without application of a magnet.

FIG. 8 is a graph that plots the breaking force required to break the silica-based shells in correlated to the thickness of the silica-based shell.

FIG. 9 is a flow chart for a method for utilizing a pressure-sensitive adhesive bandage according to an embodiment of the application.

### DETAILED DESCRIPTION

The exemplary embodiments of the present disclosure are described with respect to pressure-sensitive adhesive bandages that feature a magnetically-triggered release mechanism, a corresponding kit, and methods for utilizing the bandage. The term “bandage” is intended to cover strips, wrappings, and other material intended to support a wound dressing, as well as wound dressings themselves. The bandage may be utilized as a medical bandage for covering up skin wounds; however, the bandage may be utilized for any application that may utilize or take advantage of bandages. The pressure-sensitive adhesive bandage, kit, and methods disclosed herein provide for a magnetically-triggered release mechanism that can reduce pain, irritation, or other problems associated with removing bandages.

The pressure-sensitive adhesive bandage may include a backing and a pressure-sensitive adhesive that may be applied to the backing. The pressure-sensitive adhesive may include capsules or microcapsules, referred to herein as microcapsules for convenience, that have silica-based shells, oil containing cores that are confined by the silica-based shells, and magnetic particles that may reside in the silica-based shells or within the cores. The magnetic particles may be subjected to a magnetic force, which may cause the silica-based shells to deform, tear, or crack and thereby cause the oil to be released from the core of the shells. With the release of the oil, the oil may come into contact with the pressure-sensitive adhesive and/or the medium to which the bandage is attached (e.g. skin) or the bandage itself and cause a reduction of the peel strength. Once the peel strength is reduced, a user can remove the bandage from a surface, such as the user's own skin.

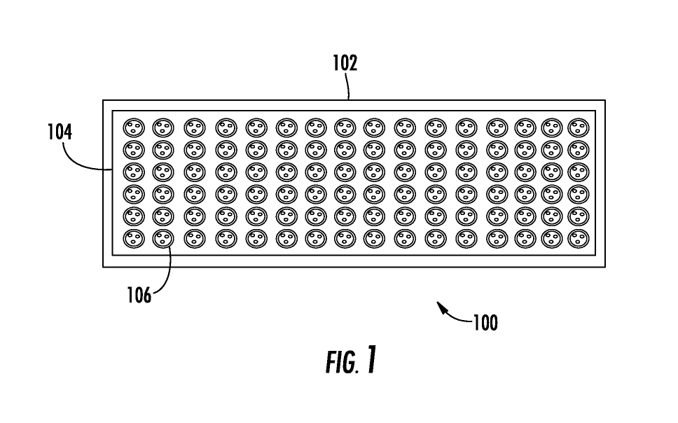

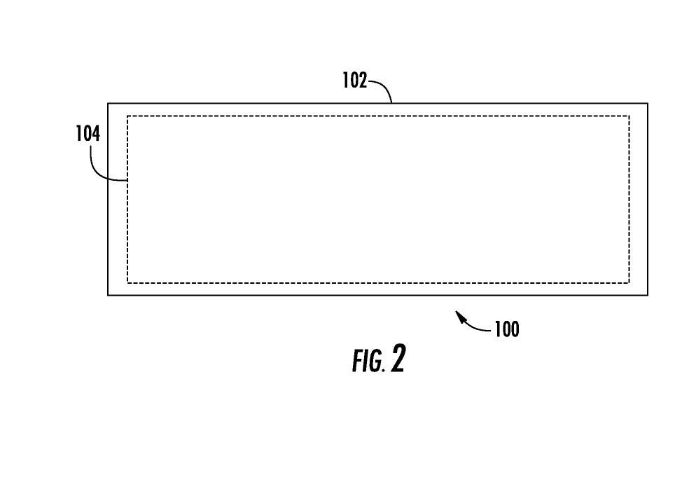

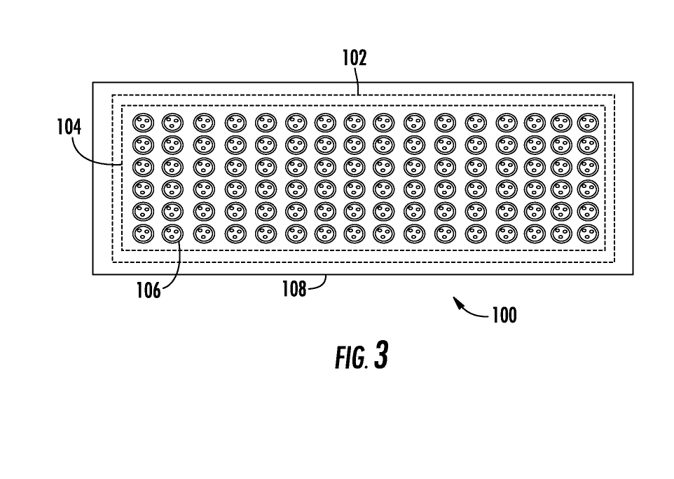

As shown in FIGS. 1-3, an exemplary pressure-sensitive adhesive bandage 100 according to an embodiment of the application is schematically illustrated. The bandage 100 may include a backing 102, a pressure-sensitive adhesive 104, a plurality of microcapsules 106, and a release liner 108. The backing 102 may be utilized as a support for the bandage 100, which may be made of, but is not limited to being made of, film, paper, plastic, polyurethane, cloth, non-wovens, vinyl, polyesters, or other similar materials.

The pressure-sensitive adhesive 104 may be applied to, or provided on, the backing 102 so that the bandage 100 can be effectively bonded or attached to a surface. In an embodiment, the pressure-sensitive adhesive 104 may be embedded in the backing 102 or may form a coating on the backing 102. The pressure-sensitive adhesive 104 may include, but is not limited to including, styrene-butadiene, acrylics, silicones, and polydimethylsiloxane (PDMS).

The release liner 108 of the bandage 100 may be utilized to cover the pressure-sensitive adhesive 104 so as to protect the pressure-sensitive adhesive 104 or otherwise prevent the pressure-sensitive adhesive 104 from adhering to objects when not in use. When the bandage 100 needs to be applied to a surface, a user may remove the release liner 108 to expose the pressure-sensitive adhesive 104, and then apply the bandage 100 to a surface, such as human skin.

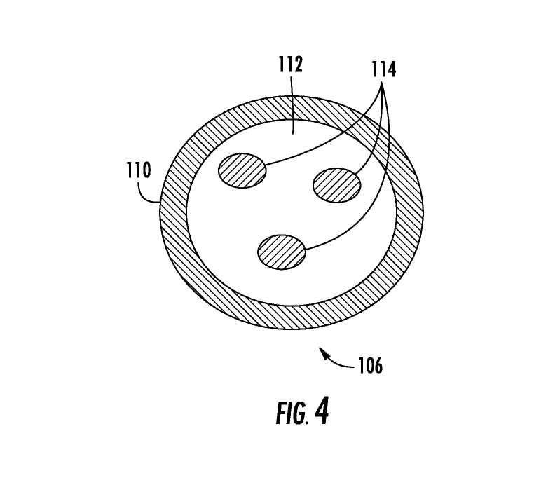

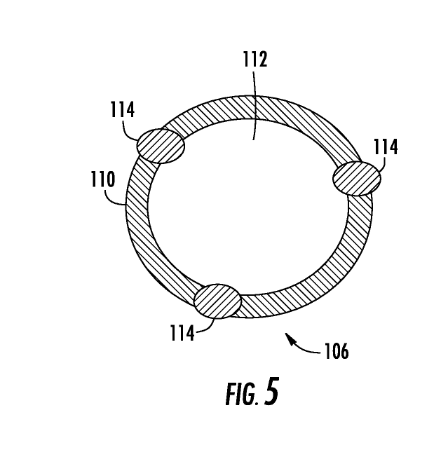

Referring now to FIGS. 4-5, the microcapsules 106 are illustrated in further detail. The microcapsules 106 may be incorporated, inserted, or embedded in, or otherwise attached to, the pressure-sensitive adhesive 104. In one embodiment, the microcapsules 106 may be located directly on or in other portions of the bandage 100. Each microcapsule 106 may include a silica-based shell 110, an oil-containing core 112, and one or more magnetic particles 114. The silica-based shell 110 of the microcapsule 106 may be formed through controlled condensation of tetraethoxysilane (TEOS) and diethoxydimethylsilane (DEODMS) onto core polydimethylsiloxane (PDMS) templates. Alternatively, the silica-based shells 110 may be created by either synthesizing emulsions of silicone oil (e.g. PDMS) from a monomer or producing the silicone oil emulsions by emulsification and then encapsulating them into the silica-based shells 110. The thickness of each of the silica-based shells 110 may be adjusted by manipulating the relative concentrations of TEOS and DEODMS or by quenching the development step of the shell. In one arrangement, the thickness of the shells is less than approximately 100 μm, in another arrangement, the thickness of the shells is less than approximately 10 μm, and in another arrangement, the thickness of the shells is less than approximately 1 μm.

The oil of core 112 may be confined by the silica-based shell 110 until a magnetic force is applied as discussed below. The oil may also be silica-based. For example, the oil may be, or may include, PDMS, polysiloxane, or other similar oils.

The magnetic particles 114 of the microcapsules 106 may be magnetic particles, microparticles, nanoparticles, or other types of magnetic particles 114, referred to herein as microparticles or nanoparticles for convenience. The type of magnetic particles 114 having the appropriate size and surface functionality along with the proper selection of the thickness for the silica-based shells 110 may be varied based on the desired application. For example, the magnetic particles 114 may include iron-oxide, nano-iron oxide, nano-magnetic particles, or other similar magnetic particles which are capable of being affected by magnetic forces. In an embodiment, the magnetic particles 114 may be inserted, embedded, and/or positioned into the silica-based shells 110. In another embodiment, the magnetic particles 114 may be positioned internally within the cores 112. In yet another embodiment, the magnetic particles 114 may be partially embedded or located in the body of the silica-based shells 110 and internally within the cores 112, or partially embedded or located in the body of the silica-based shells 110 and provide externally therefrom.

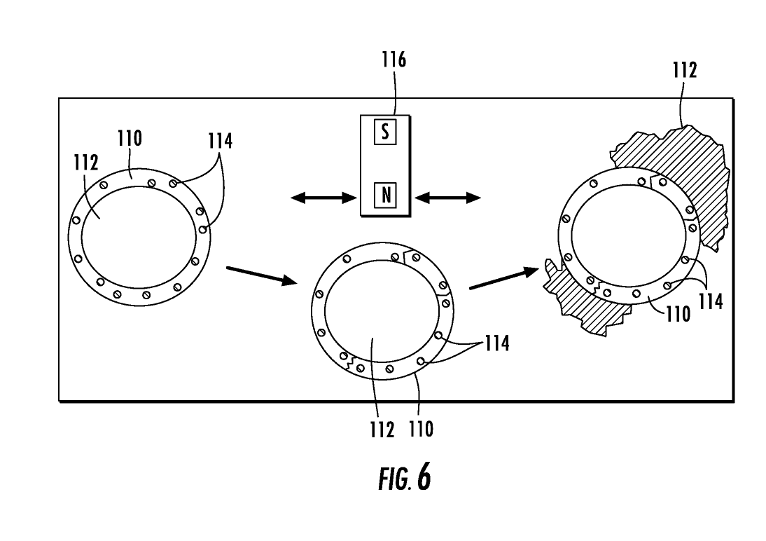

When a user decides to utilize the bandage 100, the user may remove a release liner 108 from the pressure-sensitive adhesive 104 so that the user may apply the bandage 100 to a surface, such as the user's own skin. The bandage 100 may be used to cover up a wound, serve as a dressing, or serve a variety of other functions. The bandage can be left in place for its intended duration. Once the user or physician determines that the user no longer needs to wear or use the bandage 100, a magnet 116 may be utilized to apply a magnetic force to the magnetic particles 114 in the bandage 100. FIG. 6 provides an illustrative view of the magnet's effect on the silica-based shells 110. The magnetic force generated by the magnet 116 may cause the magnetic particles 114 to deform, tear and/or crack the silica-based shells 110 in the bandage 100, thereby releasing the oil contained within the cores 112. The released oil may then contact the pressure-sensitive adhesive 104 and/or the medium to which the bandage is attached and cause a reduction in the peel strength of the pressure-sensitive adhesive 104 so that the user may remove the bandage with relative ease and minimum discomfort.

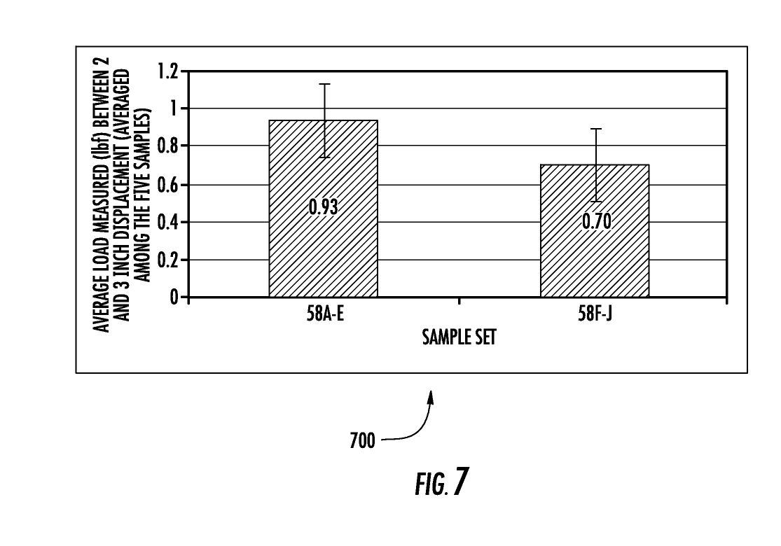

Referring now to FIG. 7, an average reduction in peel strength for test bandages having shells 110 with oil filled cores 112 and magnetic particles 114 that are either exposed or unexposed to magnet treatment is illustrated. Samples 58A-E included bandages with shells 110, cores 112 and particles 114 that were not exposed to magnetic treatment. Samples 58F-J included tape strips with shells 110, cores 112 and particles 114 that were exposed to magnetic treatment. Microcapsule samples were sprayed onto medical grade test strips, dried, applied to aluminum plates, and peel tested at 180 degrees Fahrenheit. The average pound-force (lbf) of each sample between two and three inches of displacement was calculated and then five samples in each set were averaged to provide a single value in lbf for samples with and without magnetic treatment. The determined values are plotted in FIG. 7 along with the standard deviation in the average displacements. When comparing the two averages for the samples, the standard deviation of both averages were determined to overlap, however, the averages were for one sample were not within the standard deviation of the other sample. Additionally, a T-test calculation comparing the data sets produced a P-value (the probability that the two data sets are the same) of 7.9%. A 7.9% P-value suggests that the was a 92.1% chance that the averages are different values and that there was a measurable reduction in force required to peel the tape that was exposed to magnetic treatment as compared to the tape that was not exposed to magnetic treatment.

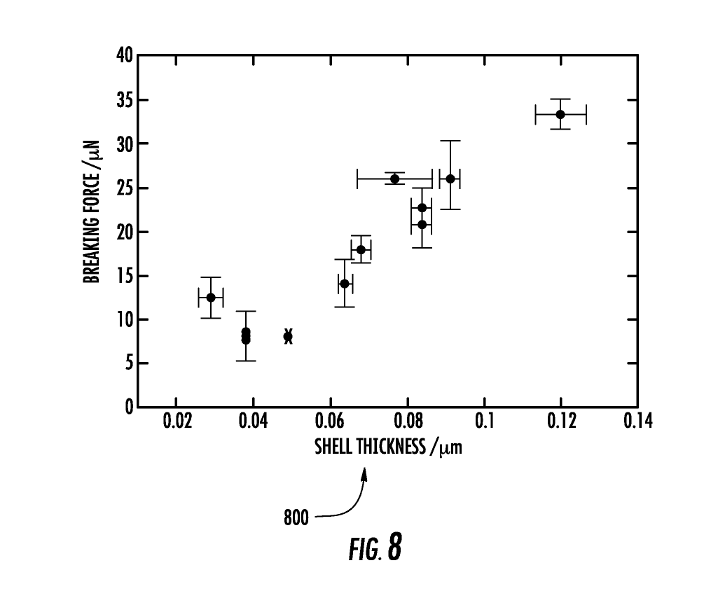

Referring now also to FIG. 8, a graph depicting the requisite breaking force in correlation to shell thickness is provided. FIG. 8 illustrates that the breaking force required to cause the silica-based shells to deform, tear and/or crack was proportional to the thickness of the silica-based shell 110. For example, at a shell thickness of 0.12 μm, the breaking force was measure as approximately 35 μN. Thus, when an appropriate magnetic force is applied to the magnetic particles 114 within a given silica-based shell 110 having a certain thickness, the magnetic force can cause the silica-based shells to release the oil from the cores 112. The released oil would then be free to contact the pressure-sensitive adhesive 104 and aid in the process of debonding the adhesive from the skin of a user or from another surface.

The pressure-sensitive adhesive bandage 100 may be provided as a kit to various users. The kit may be a package that includes a bandage 100 that includes a backing 102 and a pressure-sensitive adhesive 104 that is applied to the backing 102. The pressure-sensitive adhesive 104 can include a plurality of microcapsules 106 that can include silica-based shells 110 with cores 112 that contain oil, and a plurality of magnetic particles 114 that can be positioned in the silica-based shells 110 and/or the cores 112. Additionally, the kit may also include a magnet 116 of appropriate strength that may be utilized to apply a magnetic force on the magnetic particles 114 of the microcapsules 106. The magnetic force may cause the silica-based shells 110 to deform, tear and/or crack and thereby cause the cores 112 to release the oil onto the pressure-sensitive adhesive 104 or the medium to which the bandage is attached. The oil may cause the peel strength of the pressure-sensitive adhesive 104 to be reduced such that a user may easily remove the bandage 100 from the user's skin or other surface.

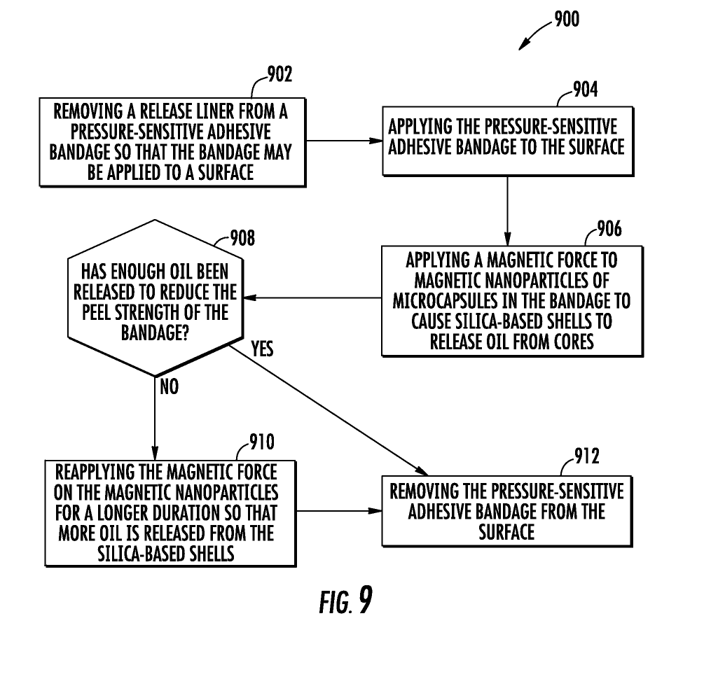

Referring now to FIG. 9, an exemplary method 900 for utilizing a pressure-sensitive adhesive bandage 100 is schematically illustrated. The method 900 may include, at step 902, removing a release liner 108 from the bandage 100 so that the bandage 100 with backing 102 and PSA 104, including silica based shells 110 with cores 112 of oil and magnetic particles 114, may be applied to a surface. At step 904 the bandage 100 can be applied to the surface, such as a user's skin.

At step 906 a magnetic force is applied to the magnetic particles 114 of the microcapsules 106 to cause the silica-based shells 110 to release the oil from the cores 112 by deforming, tearing and/or cracking the silica-based shells 110. The magnetic force may be applied by utilizing a magnet, such as magnet 116. The oil released from the cores 112 may decrease the peel strength of the pressure-sensitive adhesive 104. The method 900 may also include, at step 908, determining whether enough oil was released from the cores 112 to reduce the peel strength of the pressure-sensitive adhesive 104. If not enough oil was released from the cores 112, the method 900 may include reapplying the magnetic force to the magnetic particles 114 for a longer duration so that more oil is released from the silica-based shells 110 at step 910. Once enough oil is released to effectively reduce the peel strength of the pressure-sensitive adhesive 104, the method 900 may include removing the bandage 100 from the surface.

In an embodiment, the method 900 may include forming the silica-based shells 110 by condensation of tetraethoxysilane and diethoxydimethylsilane to form cores 112. In another embodiment, the silica-based shells 110 may be generated by synthesizing silicone oil emulsions from a monomer or by emulsification. In yet another embodiment, the magnetic particles 114 may include nano-iron oxide and nano-magnetic iron particles. Furthermore, it is important to note that the methods, devices, and kits described above may incorporate any of the functionality, components, and/or features described above or otherwise and are not intended to be limited to the description provided above.

The illustrations of arrangements described herein are intended to provide a general understanding of the structure of various embodiments, and they are not intended to serve as a complete description of all the elements and features of apparatus and methods that might make use of the structures described herein. Many other arrangements will be apparent to those of skill in the art upon reviewing the above description. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific arrangements have been illustrated and described herein, the disclosure is intended to cover any and all adaptations or variations of various embodiments and arrangements of the invention. Combinations of the above arrangements, and other arrangements not specifically described herein, will be apparent to those of skill in the art upon reviewing the above description. Therefore, it is intended that the disclosure not be limited to the particular arrangement(s) disclosed as the best mode contemplated for carrying out this invention, but that the invention will include all embodiments and arrangements falling within the scope of the appended claims.

We claim:

1. A pressure-sensitive adhesive bandage comprising:
a backing; and
a pressure-sensitive adhesive provided by the backing, the pressure-sensitive adhesive including a plurality of microcapsules, the microcapsules having:
a silica-based shell;
a core confined within the silica-based shell, wherein the core includes oil;
a plurality of magnetic particles positioned in at least one of the silica-based shell and the core;
wherein the plurality of magnetic particles are arranged such that exertion of a magnetic force releases the oil from the core; and
wherein the oil released from the core decreases peel strength of the pressure-sensitive adhesive.

2. The pressure-sensitive adhesive bandage of claim 1, wherein the magnetic particles are arranged such that the magnetic force exerted on the plurality of magnetic particles causes the silica-based shell to crack, thereby allowing the oil to be released.

3. The pressure-sensitive adhesive bandage of claim 1, wherein the silica-based shell is formed by one of synthesizing silicone oil emulsions from a monomer or by emulsification.

4. The pressure-sensitive adhesive bandage of claim 4, wherein the silicone oil emulsions include polydimethylsiloxane.

5. The pressure-sensitive adhesive bandage of claim 1, wherein the silica-based shell is formed by condensation of tetraethoxysilane and diethoxydimethylsilane onto the core.

6. The pressure-sensitive adhesive bandage of claim 1, wherein the plurality of magnetic particles include at least one of nano-iron oxide and nano-magnetic iron particles.

7. The pressure-sensitive adhesive bandage of claim 1, wherein the pressure-sensitive adhesive bandage further comprises a release liner for protecting the pressure-sensitive adhesive before the pressure-sensitive adhesive bandage is applied to the surface.

8. The pressure-sensitive adhesive bandage of claim 1, wherein the oil of the core includes polydimethylsiloxane.

9. The pressure-sensitive adhesive bandage of claim 1, wherein a thickness of the silica-based shell is determined based on concentrations of tetraethoxysilane and diethoxydimethylsilane.

10. A method for utilizing a pressure-sensitive adhesive bandage, the method comprising:
applying the pressure-sensitive adhesive bandage to a surface, wherein the pressure-sensitive bandage includes a pressure-sensitive adhesive having a plurality of microcapsules including:
a silica-based shell;
a core confined within the silica-based shell, wherein the core includes oil;
a plurality of magnetic particles positioned in at least one of the silica-based shell and the core;
wherein the plurality of magnetic particles are arranged such that exertion of a magnetic force releases the oil from the core; and
wherein the oil released from the core decreases peel strength of the pressure-sensitive adhesive.

11. The method of claim 12, wherein the pressure-sensitive adhesive bandage further comprises a release liner, and further comprising removing the release liner before applying the pressure-sensitive adhesive bandage to the surface.

12. The method of claim 12, further comprising cracking the silica-based shell when the magnetic force is applied to the plurality of magnetic particles, thereby causing the oil to be released from the core.

13. The method of claim 12, further comprising applying the magnetic force to the plurality of magnetic particles by utilizing a magnet.

14. A pressure-sensitive adhesive bandage kit comprising:
a backing;
a pressure-sensitive adhesive applied to the backing, wherein the pressure-sensitive adhesive includes a plurality of microcapsules including:
a silica-based shell;
a core confined within the silica-based shell, wherein the core includes oil;
a plurality of magnetic particles positioned in at least one of the silica-based shell and the core;
wherein the plurality of magnetic particles are arranged such that exertion of a magnetic force releases the oil from the core;
wherein the oil released from the core decreases peel strength of the pressure-sensitive adhesive; and
a magnet for removal of the adhesive bandage after use.

15. The pressure sensitive-adhesive bandage kit of claim 14, comprising sets of bandages of different size, and sets of magnets of different strengths.
